(19) United States
(12) Patent Application Publication
(10) Pub. No.: US 2025/0235649 A1
(43) Pub. Date: Jul. 24, 2025

(54) NASAL MASK

(71) Applicant: HSINER CO., LTD., Taichung City (TW)

(72) Inventor: Eric CHANG, Taichung City (TW)

(21) Appl. No.: 18/658,643

(22) Filed: May 8, 2024

(30) Foreign Application Priority Data
Jan. 24, 2024 (TW) 113102749

(51) Int. Cl. A61M 16/06; A61M 16/08

(52) U.S. Cl. CPC A61M 16/0622; A61M 16/0816; A61M 16/0875; A61M 2210/0618

(57) ABSTRACT

A nasal mask includes a frame, a cushion seal and a flexible tube. The frame includes a frame body and a socket extends therethrough. The socket has a first inner diameter on a first sectioned plane perpendicular to a left-right direction, and a second inner diameter on a second sectioned plane perpendicular to an up-down direction. The second inner diameter is larger than the first inner diameter. The seal includes a seal body. The flexible tube is made of a flexible material and includes a connector portion deformably and pluggably mounted in the socket so as to conveniently mount the flexible tube on the frame.

### CROSS-REFERENCE TO RELATED APPLICATION

This application claims priority to Taiwanese Patent Application No. 113102749, filed on Jan. 24, 2024, and incorporated by reference herein in its entirety.

### FIELD

The disclosure relates to a nasal mask, and more particularly to a nasal mask used for delivering pressurized gases to a person being treated for obstructive sleep apnea (OSA) or other disorders.

### BACKGROUND

Referring to FIG. 1, a conventional nasal interface disclosed in TW 201627027A includes a frame unit 91, a seal unit 92 and a gas delivering unit 93. The frame unit 91 is made of a rigid material, and includes a frame body 911, a sleeve ring 912 formed on a rear side of the frame body 911, and a surrounding flange 913 formed on a front side of the frame body 911 to cooperate with the sleeve ring 912 to define an intake hole 914. A surrounding groove 915 is formed in an outer peripheral wall of the surrounding flange 913. The seal unit 92 is made of a flexible material, and includes a front wall 921, a rear wall 922, a sleeve hole 923 formed in the front wall 921 to be engaged with the sleeve ring 912, and two nostril locators 924 formed on and extending rearwardly from the rear wall 922. The gas delivering unit 93 includes a tubular coupler 931 removably mounted on the surrounding flange 913, and a gas delivering tube 932 rotatably mounted on the tubular coupler 931. The tubular coupler 931 has an insert portion 933 inserted into the surrounding flange 913, and two flexible barbs 934 disposed on left and right sides of the insert portion 933 to be clipped into the surrounding groove 915. The gas delivering tube 932 has an end inserted into the insert portion 933, and two end stoppers 935 (only one is shown in FIG. 1) disposed rearwardly of the insert portion 933 so as to retain the gas delivering tube 932 to the tubular coupler 931.

However, when the patient wearing the nasal interface turns over the nasal interface may be pulled, the tubular coupler 931 which interconnects the gas delivering tube 932 and the frame body 911 may be worn and could cause gas leakage, and hence is needed to be replaced regularly, which increases the part cost. Moreover, when it is desired to remove the gas delivering tube 932 for washing, the tubular coupler 931 must be removed with great effort due to the rigid end stoppers 935 and the insert portion 933, which results in inconvenience during the removal operation.

### SUMMARY

Therefore, an object of the disclosure is to provide a nasal mask that can be separated conveniently and has decreased part costs.

According to the disclosure, the nasal mask includes a frame, a cushion seal and a flexible tube. The frame is made of a rigid material, and includes a frame body, a socket which extends through the frame body in a front-rear direction, and a rear surrounding wall which extends rearwardly from the frame body and which surrounds the socket. The frame body has a surrounding flange which surrounds a front end of the socket. The socket has a first inner diameter which is measured on a first sectioned plane that is perpendicular to a left-right direction, and a second inner diameter which is measured on a second sectioned plane that is perpendicular to an up-down direction and which is larger than the first inner diameter. The front-rear direction, the left-right direction and the up-down direction are perpendicular to one another. The cushion seal is made of a flexible material, and is removably mounted on the frame. The cushion seal includes a seal body which has a front wall and a rear wall that are spaced apart from each other in the front-rear direction to define a chamber therebetween, a sleeve hole which is formed in and extends through the front wall in the front-rear direction and which is engaged with the rear surrounding wall, and an intake hole which is formed in and extends through the rear wall in the front-rear direction to be communicated with the sleeve hole through the chamber. The flexible tube is made of a flexible material, and is in communication with the chamber. The flexible tube includes a tube portion and a connector portion which is connected with the tube portion and which is deformably and pluggably mounted in the socket. The connector portion has an outer peripheral wall and a mounting surrounding groove which is formed in the outer peripheral wall. The mounting surrounding groove has a first groove width which is measured on the first sectioned plane, and a second groove width which is measured on the second sectioned plane. The connector portion is deformable and shifted between a non-mounted state, where the flexible tube is disengaged from the frame, and a mounted state, where the flexible tube is connected with the frame. When the connector portion is in the non-mounted state, the first groove width is larger than the first inner diameter, and the second groove width is smaller than the second inner diameter. When the connector portion is in the mounted state, the first groove width is smaller than the first inner diameter such that the connector portion is deformed and expanded in the left-right direction to permit fitting engagement of the surrounding flange in the mounting surrounding groove.

With the socket having the second inner diameter larger than the first inner diameter, and the flexible tube made of a flexible material, the connector portion is deformable and inserted into the socket so as to be conveniently mounted on and removed from the frame. Moreover, connecting means between the flexible tube and the frame is optimized so as to reduce the number of parts and assembling costs.

### BRIEF DESCRIPTION OF THE DRAWINGS

Other features and advantages of the disclosure will become apparent in the following detailed description of the embodiment with reference to the accompanying drawings. It is noted that various features may not be drawn to scale.

FIG. 1 is an exploded perspective view of a conventional nasal interface.

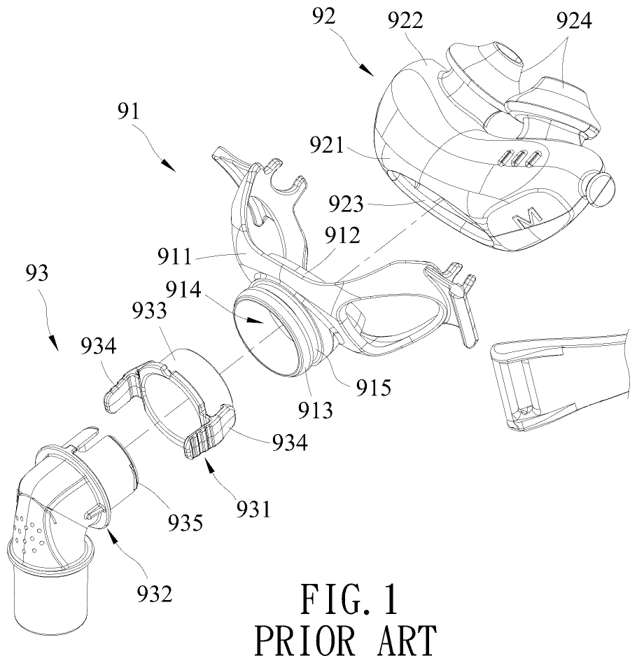

FIG. 2 is a perspective view illustrating an embodiment of a nasal mask according to the disclosure.

FIG. 3 is an exploded perspective view of the embodiment.

FIG. 4 is an exploded perspective view of the embodiment taken from another angle.

FIG. 5 is a front view illustrating a frame and a cushion seal of the embodiment.

FIG. 6 is a partly sectioned view of FIG. 5.

FIG. 7 is a fragmentary, exploded sectional view of the embodiment.

FIG. 8 is a fragmentary sectional view of the embodiment.

FIG. 9 is a fragmentary, exploded, sectional top view of the embodiment.

FIG. 10 is a fragmentary, sectional top view of the embodiment.

### DETAILED DESCRIPTION

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

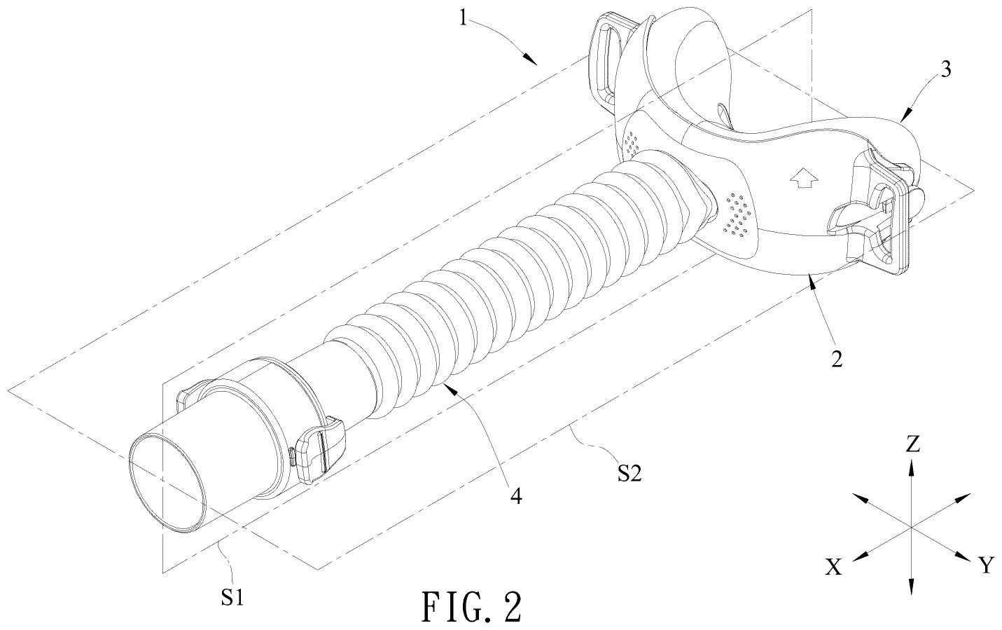

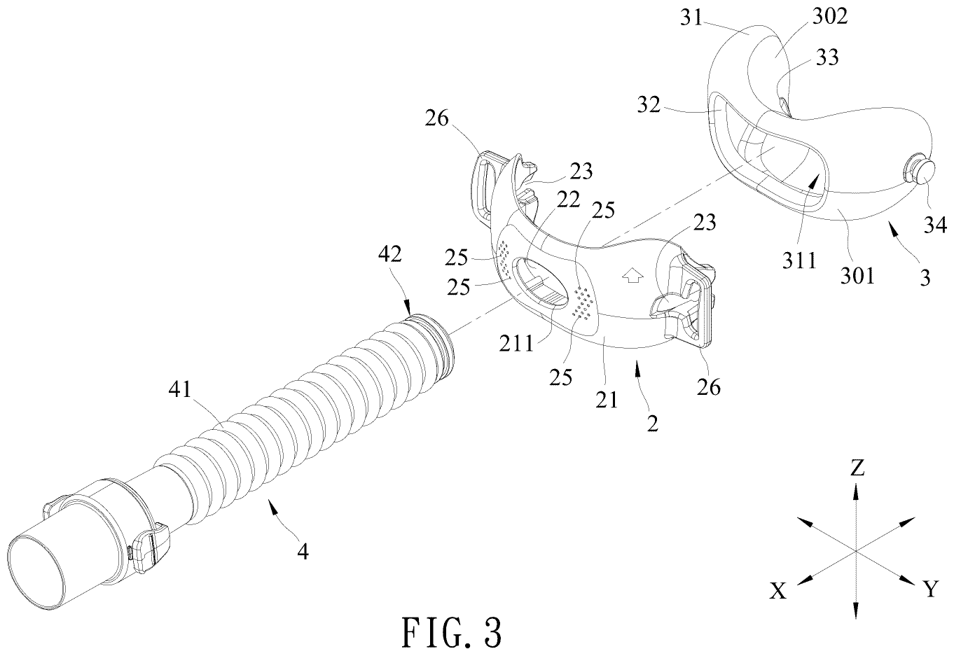

Referring to FIGS. 2 and 3, an embodiment of a nasal mask 1 according to the disclosure includes a frame 2, a cushion seal 3 and a flexible tube 4.

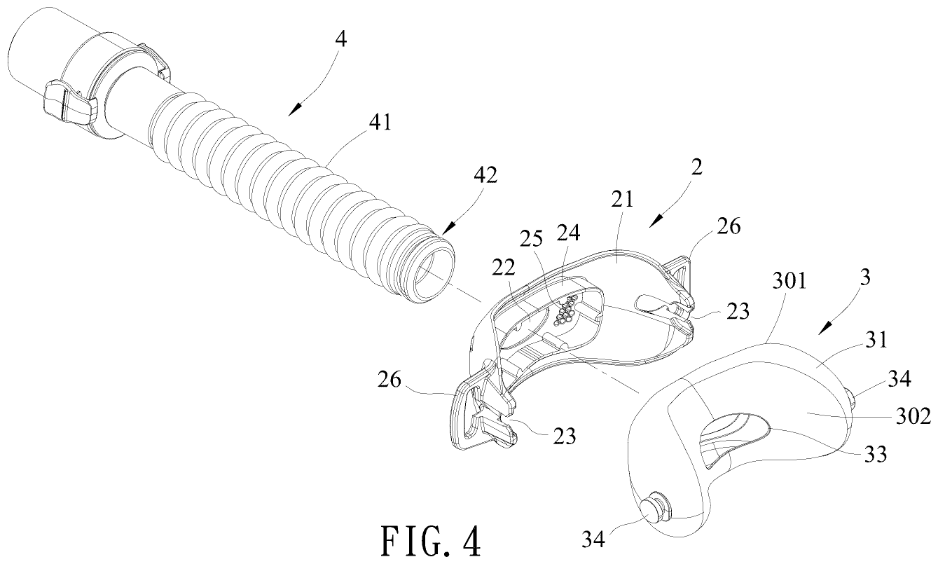

With reference to FIGS. 3 to 5, the frame 2 is made of a rigid material, and includes a frame body 21, a socket 22 which extends through the frame body 21 in a front-rear direction (X), two positioning recesses 23 which are formed in the frame body 21 and at left and right sides of the frame body 21 and which are opened rearwardly, a rear surrounding wall 24 which extends rearwardly from the frame body 21 and which surrounds the socket 22, a plurality of bias flow apertures 25 which extend through the frame body 21 in the front-rear direction (X) and which are formed at left and right sides of the socket 22, and two lugs 26 which extend laterally from the left and right sides of the frame body 21 for connecting two ends of a head strap (not shown) that can pass around the back of a patient's head.

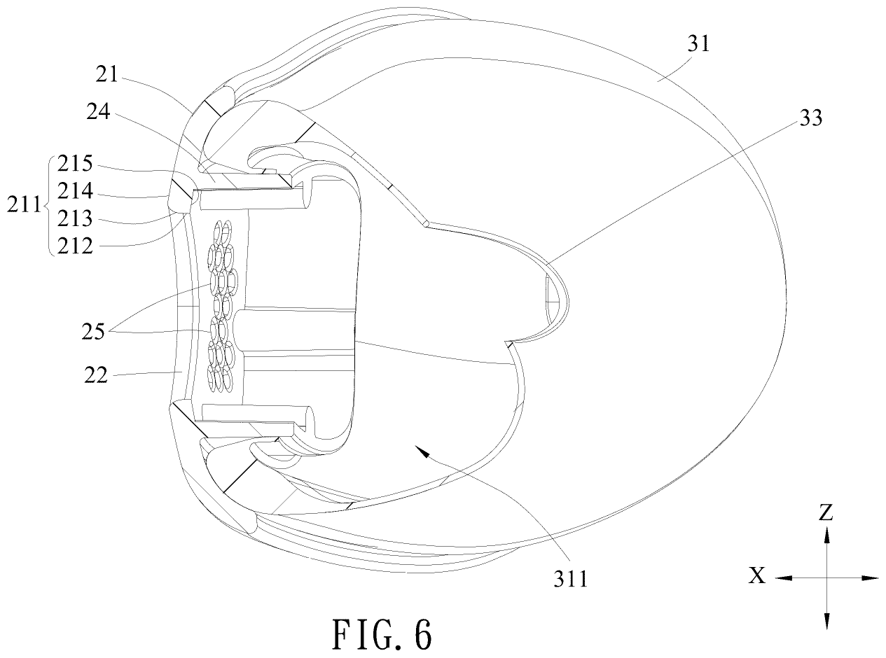

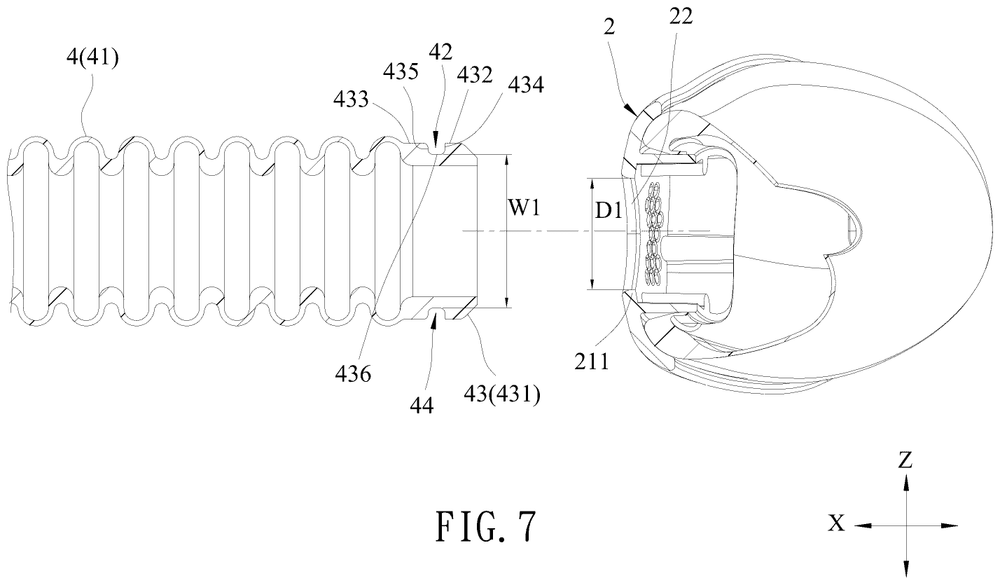

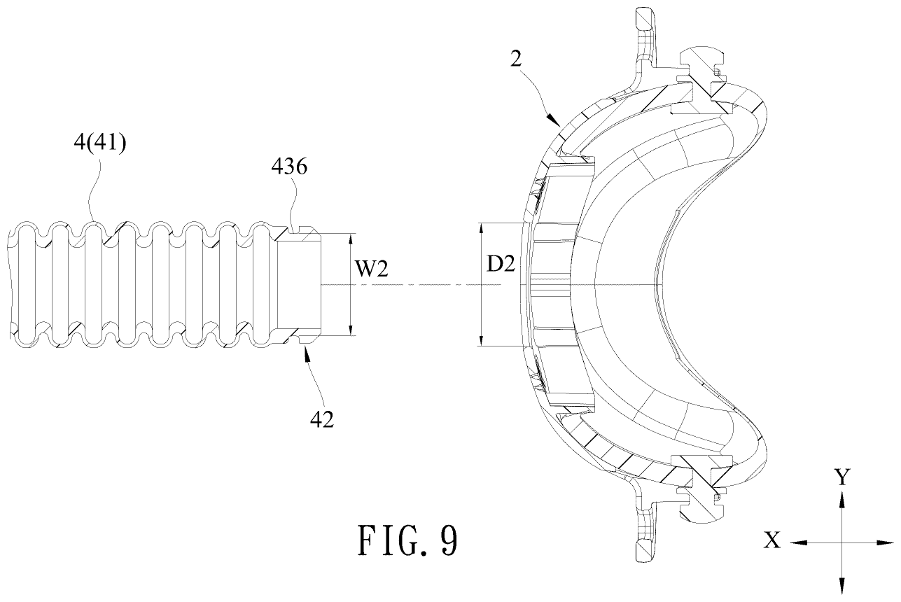

With reference to FIGS. 3 and 6, the frame body 21 has a surrounding flange 211 which surrounds a front end of the socket 22. With reference to FIGS. 2, 5 and 6, the socket 22 is in the form of an oval hole, and has a first inner diameter (D1) (see FIG. 7) which is measured on a first sectioned plane (S1) that is perpendicular to a left-right direction (Y), and a second inner diameter (D2) (see FIG. 9) which is measured on a second sectioned plane (S2) that is perpendicular to an up-down direction (Z). The second inner diameter (D2) is larger than the first inner diameter (D1). A ratio of the first inner diameter (D1) to the second inner diameter (D2) ranges from 0.58 to 0.62. The front-rear direction (X), the left-right direction (Y) and the up-down direction (Z) are perpendicular to one another.

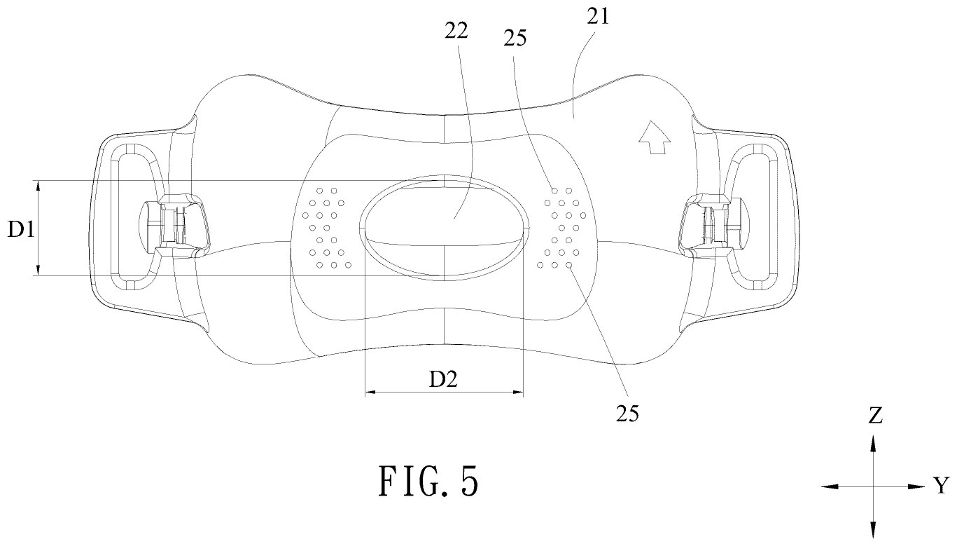

With reference to FIGS. 5 and 6, the surrounding flange 211 of the frame body 21 has a flat end surface section 212 which defines the first inner diameter (D1) and the second inner diameter (D2), a flared end surface section 213 which is connected with and formed forwardly of the flat end surface section 212, a forward facing surface section 214 which is connected with the flared end surface section 213, and a rearward facing surface section 215 which is opposite to the forward facing surface section 214 in the front-rear direction (X) and which is connected with the flat end surface section 212. The rearward facing surface section 215 extends and is inclined rearwardly from the flat end surface section 212 to the rear surrounding wall 24.

With reference to FIGS. 3, 4 and 6, the cushion seal 3 is made of a flexible material, and is removably mounted on the frame 2. The cushion seal 3 includes a seal body 31 which has a front wall 301 and a rear wall 302 that are spaced apart from each other in the front-rear direction (X) to define a chamber 311 therebetween, a sleeve hole 32 which is formed in and extends through the front wall 301 in the front-rear direction (X) and which is engaged with the rear surrounding wall 24, an intake hole 33 which is formed in and extends through the rear wall 302 in the front-rear direction (X) to be communicated with the sleeve hole 32 through the chamber 311, and two barbs 34 which are formed on left and right sides of the seal body 31 to be extended forwardly and clipped into the positioning recesses 23, respectively. The bias flow apertures 25 provide flow paths from the chamber 311 to ambient surroundings.

With reference to FIGS. 3, 4 and 8, the flexible tube 4 is made of a flexible material, and is in communication with the chamber 311. The flexible tube 4 includes a tube portion 41 and a connector portion 42 connected with the tube portion 41. The connector portion 42 is deformably and pluggably mounted in the socket 22. With reference to FIG. 7, the connector portion 42 has an outer peripheral wall 43 and a mounting surrounding groove 44 formed in the outer peripheral wall 43. The outer peripheral wall 43 has a surrounding inclined section 431 which is formed at an end thereof opposite to the tube portion 41, a first connecting section 432 which is connected with and formed forwardly of the surrounding inclined section 431, a second connecting section 433 which is connected with and formed rearwardly of the tube portion 41, a first abutting section 434 which extends radially and inwardly from the first connecting section 432, a second abutting section 435 which extends radially and inwardly from the second connecting section 433, and a third abutting section 436 which interconnects the first abutting section 434 and the second abutting section 435 to cooperatively define the mounting surrounding groove 44. With reference to FIGS. 2, 7 and 9, the third abutting section 436 has a first groove width (W1) (see FIG. 7) which is measured on the first sectioned plane (S1), and a second groove width (W2) (see FIG. 9) which is measured on the second sectioned plane (S2).

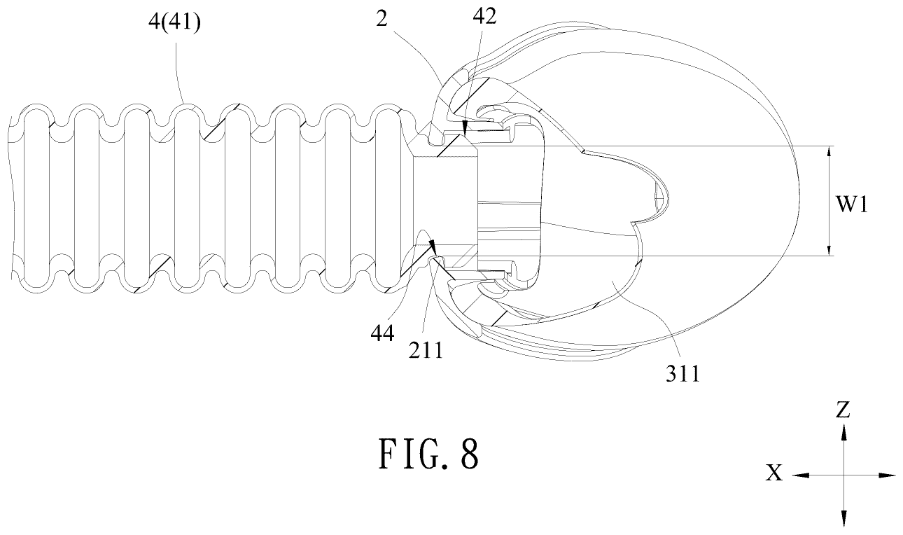

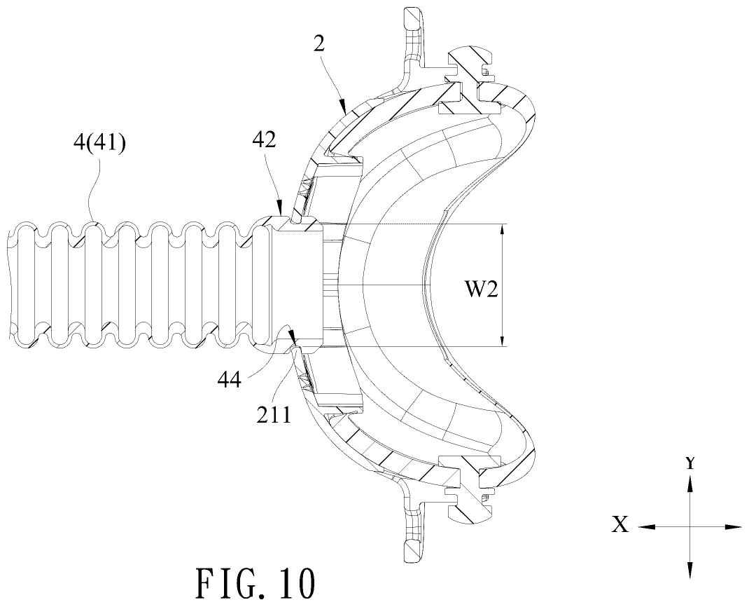

The connector portion 42 is operable to be deformable and shifted between a non-mounted state, as shown in FIGS. 7 and 9, where the flexible tube 4 is disengaged from the frame 2, and a mounted state, as shown in FIGS. 8 and 10, where the flexible tube 4 is connected with the frame 2. When the connector portion 42 is in the non-mounted state, the first groove width (W1) is larger than the first inner diameter (D1), and the second groove width (W2) is smaller than the second inner diameter (D2). When the connector portion 42 is in the mounted state, the connector portion 42 is compressed and deformed in the up-down direction (Z) while expanded in the left-right direction (Y) so as to permit fitting engagement of the surrounding flange 211 in the mounting surrounding groove 44. In this state, the first abutting section 434 is in abutting engagement with the rearward facing surface section 215, the second abutting section 435 is in abutting engagement with the forward facing surface section 214, the third abutting section 436 is in abutting engagement with the flat end surface section 212 and the flared end surface section 213, the first groove width (W1) is slightly smaller than but approximate to the first inner diameter (D1), and the second groove width (W2) is slightly smaller than but approximate to the second inner diameter (D2).

It is noted that, with the flexible tube 4 made of a flexible material, the connector portion 42 is compressed and deformed in the up-down direction (Z) in the mounted state to generate a biasing force in the up-down direction (Z), which permits the third abutting section 436 to tightly abut against the flat end surface section 212 so that the surrounding flange 211 is firmly retained in the mounting surrounding groove 44.

With reference to FIGS. 6 to 8, when it is desired to mount the flexible tube 4 on the frame 2, the operator applies a compression force in the up-down direction (Z) to the connector portion 42 to compress and deform the connector portion 42 in the up-down direction (Z) while expanding the connector portion 42 in the left-right direction (Y) so as to shape the connector portion 42 into an oval profile corresponding with the socket 22. Then, the operator applies a press force in the front-rear direction (X) to the connector portion 42 with the surrounding inclined section 431 abutting against the flared end surface section 213 such that the surrounding flange 211 is moved to engage the surrounding inclined section 431 and the first connecting section 432. At this stage, the connector portion 42 is compressed and deformed again in the up-down direction (Z) while being expanded in the left-right direction (Y) until the surrounding flange 211 is engaged in the mounting surrounding groove 44 to bring the connector portion 42 in the mounted state.

The surrounding inclined section 431 abuts against and is moved over the flared end surface section 213 during the mounting operation, which facilitates insertion of the connector portion 42 into the socket 22.

The operation of removing the flexible tube 4 from the frame 2 is described as follows.

The connector portion 42 is compressed and deformed with a compression force in the up-down direction (Z) to disengage the surrounding flange 211 from the mounting surrounding groove 44 in the up-down direction (Z) while keeping the engagement of the surrounding flange 211 with the mounting surrounding groove 44 in the left-right direction (Y). Then, a pulling force is applied to the flexible tube 4 to forcedly remove the flexible tube 4 from the frame 2. With reference to FIGS. 6 and 7, specifically, with the rearward facing surface section 215 extending and inclined rearwardly from the flat end surface section 212 to the rear surrounding wall 24, and the flexible tube 4 made of a flexible material, during the pulling operation of the flexible tube 4 by the pulling force, the first abutting section 434 is moved along the rearward facing surface section 215 to have the surrounding inclined section 431 be bent radially and inwardly so as to permit removal of the surrounding flange 211 from the mounting surrounding groove 44.

Alternatively, a greater pulling force may be applied directly to the flexible tube 4 to remove the flexible tube 4 from the frame 2. According to the experimentally tested result of the nasal mask 1 of the disclosure, when the connector portion 42 is in the mounted state, the connector portion 42 is removable from the frame 2 with a first pulling force which is in the front-rear direction (X) and which is at least 2.2 kgf, with a second pulling force which is applied on the second sectioned plane (S2) and intersects the left-right direction (Y) by an angle of 45 degrees and which is at least 1.4 kgf, or with a third pulling force which is applied on the first sectioned plane (S1) and intersects the front-rear direction (X) by an angle of 45 degrees and which is at least 0.6 kgf. According to the experimental test, when a user wearing the nasal mask 1 sleeps, the maximum pulling force exerted on the flexible tube 4 due to turning over is less than 0.6 kgf. Hence, an undesired removal of the flexible tube 4 from the frame 2 is avoided during sleeping and wearing the nasal mask 1.

As illustrated, with the socket 22 having the second inner diameter (D2) larger than the first inner diameter (D1), and the flexible tube 4 being made of a flexible material, the connector portion 42 is deformable and inserted into the socket 22 so as to be conveniently mounted on and removed from the frame 2. Moreover, it is not required to increase connecting means between the flexible tube 4 and the frame 2 so as to reduce the part and assembling costs.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A nasal mask, comprising:
a frame made of a rigid material, and including a frame body, a socket which extends through said frame body in a front-rear direction, and a rear surrounding wall which extends rearwardly from said frame body and which surrounds said socket, said frame body having a surrounding flange which surrounds a front end of said socket, said socket having a first inner diameter which is measured on a first sectioned plane that is perpendicular to a left-right direction, and a second inner diameter which is measured on a second sectioned plane that is perpendicular to an up-down direction and which is larger than the first inner diameter, the front-rear direction, the left-right direction and the up-down direction being perpendicular to one another;
a cushion seal made of a flexible material, and removably mounted on said frame, said seal including a seal body which has a front wall and a rear wall that are spaced apart from each other in the front-rear direction to define a chamber therebetween, a sleeve hole which is formed in and extends through said front wall in the front-rear direction and which is engaged with said rear surrounding wall, and an intake hole which is formed in and extends through said rear wall in the front-rear direction to be communicated with said sleeve hole through said chamber; and
a flexible tube made of a flexible material, and in communication with said chamber, said flexible tube including a tube portion and a connector portion which is connected with said tube portion and which is deformably and pluggably mounted in said socket, said connector portion having an outer peripheral wall and a mounting surrounding groove which is formed in said outer peripheral wall, said mounting surrounding groove having a first groove width which is measured on the first sectioned plane, and a second groove width which is measured on the second sectioned plane, said connector portion being deformable and shifted between a non-mounted state, where said flexible tube is disengaged from said frame, and a mounted state, where said flexible tube is connected with said frame, wherein, when said connector portion is in the non-mounted state, the first groove width is larger than the first inner diameter, and the second groove width is smaller than the second inner diameter, and wherein, when said connector portion is in the mounted state, the first groove width is smaller than the first inner diameter such that said connector portion is deformed and expanded in the left-right direction to permit fitting engagement of said surrounding flange in said mounting surrounding groove.

2. The nasal mask of claim 1, wherein said outer peripheral wall of said connector portion has a surrounding inclined section which is formed at an end thereof opposite to said tube portion, said surrounding flange of said frame body having a flat end surface section which defines the first inner diameter and the second inner diameter, and a flared end surface section which is connected with and formed forwardly of said flat end surface section.

3. The nasal mask of claim 2, wherein said surrounding flange of said frame body further has a forward facing surface section connected with said flared end surface section, and a rearward facing surface section connected with said flat end surface section, said outer peripheral wall of said connector portion further having a first connecting section which is connected with and formed forwardly of said surrounding inclined section, a second connecting section which is connected with and formed rearwardly of said tube portion, a first abutting section which extends radially and inwardly from said first connecting section, a second abutting section which extends radially and inwardly from said second connecting section, and a third abutting section which interconnects said first abutting section and said second abutting section to cooperatively define said mounting surrounding groove, said third abutting section having the first groove width on the first sectioned plane, and the second groove width on the second sectioned plane, wherein, when said connector portion is in the mounted state, said first abutting section is in abutting engagement with said rearward facing surface section, said second abutting section is in abutting engagement with said forward facing surface section, and said third abutting section is in abutting engagement with said flat end surface section and said flared end surface section.

4. The nasal mask of claim 1, wherein, when said connector portion is in the mounted state, the first groove width is smaller than and approximate to the first inner diameter, and the second groove width is smaller than and approximate to the second inner diameter.

5. The nasal mask of claim 1, wherein said frame further includes a plurality of bias flow apertures extending through said frame body in the front-rear direction to provide flow paths from said chamber to ambient surroundings, said bias flow apertures being formed at left and right sides of said socket.

6. The nasal mask of claim 1, wherein said frame further includes two positioning recesses which are formed in said frame body and at left and right sides of said frame body and which are opened rearwardly, said cushion seal further including two barbs which are formed on left and right sides of said seal body to be extended forwardly and clipped into said positioning recesses, respectively.

7. The nasal mask of claim 1, wherein a ratio of the first inner diameter to the second inner diameter ranges from 0.58 to 0.62.

8. The nasal mask of claim 1, wherein, when said connector portion is in the mounted state, said connector portion is removable from said frame with a first pulling force which is in the front-rear direction and which is at least 2.2 kgf, with a second pulling force which is on the second sectioned plane and intersects the left-right direction by an angle of 45 degrees and which is at least 1.4 kgf, or with a third pulling force which is on the first sectioned plane and intersects the front-rear direction by an angle of 45 degrees and which is at least 0.6 kgf.
